(19) United States
(12) Patent Application Publication
(10) Pub. No.: US 2009/0076375 A1
(43) Pub. Date: Mar. 19, 2009

(54) Myocardial tissue ablation device for treatment of cardiac arrhythmias by ablation of myocardial tissue in a patient as well as associated catheter and associated method

(75) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 12/283,416

(22) Filed: Sep. 11, 2008

(30) Foreign Application Priority Data
Sep. 13, 2007 (DE) 10 2007 043 732.5

(51) Int. Cl. A61B 1/00; A61B 6/12; A61B 5/055

(52) U.S. Cl. 600/411; 600/104; 600/424

(57) ABSTRACT

The invention relates to a myocardial tissue ablation device for treatment of cardiac arrhythmias by ablation of myocardial tissue in a patient, with the myocardial tissue ablation device featuring a catheter which is embodied as an integrated unit with at least one myocardial ablation means and at least one imaging element based on optical coherence tomography and/or intravascular magnetic resonance imaging.

### CROSS REFERENCE TO RELATED APPLICATIONS

This application claims priority of German application No. 10 2007 043 732.5 filed Sep. 13, 2007, which is incorporated by reference herein in its entirety.

### FIELD OF THE INVENTION

The invention relates to a myocardial tissue ablation device for treatment of cardiac arrhythmias by ablation of myocardial tissue in a patient.

### BACKGROUND OF THE INVENTION

Cardiac arrhythmias are problems with the normal heartbeat caused by problems in excitation generation or conduction in the myocardium.

One such is bradycardia, in which a reduction of the heart frequency is present. Heart pacemakers, which are intended to re-establish the normal sine-wave rhythm, have been used for many years to treat this condition. In addition there are tachycardia arrhythmias such as atrial fibrillation for example. With these problems the atrium is excited at high frequency by conduction path problems in the heart. With ventricular tachycardias the problem results in there no longer being a complete contraction of the heart, caused by a lack of pumping power of the heart. Therapy measures to counter the occurrence of tachycardias are for example an ongoing course of medicines or a heart operation in which the conduction path tissue is cut away in specific parts of the heart. The disadvantage in such cases is the relatively high risk or patient.

Minimally-invasive therapy methods are thus increasingly being used to fight cardiac arrhythmias. These involve introducing an ablation catheter via a vein entry into the area of the heart in order to destroy faulty conduction paths there, for example by using the effect of energy to “burn them away”. A prerequisite for this is that the conduction paths and points causing the problem are known and are able to be targeted directly by the catheter. This method is used for example with supraventricular tachycardias, but also increasingly in the area of ventricular tachycardias, since treatment without medicaments with antiarrhythmic drugs only has a very low success rate and on the other hand implantable defibrillators have unpleasant side effects for the patient. However it remains difficult to identify or to target the correct locations for ablation during the interval.

Methods are known in which the electrophysiological potentials in the heart are established for determining the correct ablation point in each case. Establishing the electrophysiological potentials is also known as mapping. Such mapping is sometimes used in combination with x-ray systems.

The electrophysiological potentials do however make it possible to actually show the tissue, especially to show it within the framework of the scar tissue arising for example as part of a coronary infarction in the heart. The origin of typical ventricular tachycardias lies however in the so-called “reentrant circuits”, which usually arise at and in the border of the electrically non-active myocardial scar tissue, so that the lack of tissue display leads to problems.

### SUMMARY OF THE INVENTION

The underlying object of the invention is thus to specify a myocardial tissue ablation device improved in this respect for treatment of heart rhythm problems by ablation of myocardial tissue in a patient.

To achieve this objective a myocardial tissue ablation device of this type is provided, which is characterized by the myocardial tissue ablation device featuring a catheter which is embodied as an integrated unit with at least one myocardial tissue ablation means and at least one imaging element based on optical coherence tomography and/or intravascular magnetic resonance imaging.

The myocardial tissue ablation device thus features, in addition to possible further components such as an x-ray imaging device or options for recording ECGs and such like and possibly a control device or console, a catheter which not only has one or more means for ablation of (e.g. scarred) myocardial tissue that is responsible for a disturbed conduction path and thereby for cardiac arrhythmias, but in addition comprises at least one integrated imaging element for creating images in accordance with the principle of optical coherence tomography (OCT) and/or intravascular magnetic resonance imaging (IVMRI).

Such a catheter for ablation of tissue responsible for disturbed conduction paths in the heart, or which is a factor in the condition, has not thus far been available in one unit with an imaging element.

This integrated catheter however offers the advantage, that during the treatment, i.e. during tissue ablation or directly before it, after it and in intervals during the ablation, images of the area involved can be created with the corresponding imaging element or also with a number of imaging elements of the catheter.

For example within the framework of magnetic resonance imaging scar tissue can be shown that has been produced during a coronary infarction for example. The IVMRI images in this case are functional and simultaneously anatomical or anatomically usable images. The integration of the IVMRI element into the catheter for the ablation of the myocardial tissue allows the magnetic resonance images to be created directly in the area of tissue removal and also during the ablation procedure.

The magnetic field required for magnetic resonance imaging can for example, in accordance with publication U.S. Pat. No. 6,377,048, be created outside the body as a static magnetic field. However a solution is preferable in which the static magnetic field for the IVMRI images is created by a catheter, i.e. inventively by the myocardial tissue ablation catheter and no external magnetic field is necessary for field generation. Generation of a magnetic field by a catheter is described in U.S. Pat. No. 6,600,319.

IVMRI can be undertaken in this case so that the corresponding catheter, i.e. the catheter of the myocardial tissue ablation device in this case with the additional ablation means, can be introduced under x-ray control into corresponding blood vessels and can be withdrawn by hand from the vessels. The use of IVMRI with its especially good soft tissue imaging for the identification of infarction scar tissue or generally in the area of the treatment of arrhythmias represents a completely new area in this case.

As an alternative or in addition at least one further imaging element based on optical coherence tomography can be integrated into the myocardial tissue ablation catheter. The basic principle of optical coherence tomography is based on the Michelson interferometer. The OCT method has the advantage of allowing tissue to be shown at high resolution to a penetration depth of several millimeters. This penetration depth is sufficient to differentiate between scar tissue and healthy tissue. The use of OCT in the area of identifying infarction scar tissue or generally in the area the treatment of cardiac arrhythmias, i.e. in conjunction with a myocardial ablation catheter or a corresponding device, is an entirely new area.

Thus the inventive integrated catheter or the corresponding myocardial tissue ablation device offers the advantage that the integration of the imaging element based on optical coherence tomography or intravascular magnetic resonance imaging with a single instrument, i.e. a single catheter, allows both ablation of the damaged tissue or of the scar tissue, which is the origin of the incorrect conduction path in the heart, and also imaging to accompany the ablation treatment. In such cases imaging based on optical coherence tomography or intravascular magnetic resonance imaging offers the advantage of not just making it possible to show tissue at all, but especially to provide a good presentation of scar tissue.

In such cases the images are recorded at a sufficient resolution and also with a corresponding depth such that not only the tissue surface, but also areas further below the surface are able to be recorded or presented.

Thus this imaging method, previously only known in the area of treatment of arteriosclerotic plaque, is also able to be used within the framework of a myocardial tissue ablation device in accordance with the invention to obtain advantageous imaging to support treatment (of cardiac arrhythmia problems).

At least one myocardial tissue ablation means can inventively be a means for emitting high-frequency and/or ultrasound and/or cold and/or heat energy.

The myocardial tissue is preferably ablated with the aid of electrical energy, especially with high-frequency radiation. It is however also possible, as an alternative or in addition, to use further modulation methods, operating on the basis of ultrasound, cold or heat.

A myocardial tissue ablation means for emitting high-frequency radiation can expediently be a high-frequency ablation coil. This can for example be arranged in front of or behind a sensor for recording data within the framework of optical coherence tomogaphy (OCT sensor) or an IVMRI sensor. If necessary the imaging sensor or another element representing a front component of the imaging element, for creating images in a region in front of the ablation means, can be passed through a corresponding opening of the ablation coil for example.

Furthermore the heart muscle tissue ablation device can feature a control device. This control device, for example in the form a console or a computing device, if necessary with a number of processing units, is then used to control the introduction of the catheter or to carry out the ablation and/or for creating the OCT or IVMRI-based images.

The control device can be embodied for automatic evaluation of at least one recorded image of at least one imaging element and/or for identifying scar tissue, especially coronary infarction scar tissue, and/or damaged conduction path tissue by means of at least one program means.

The control device thus has a memory area for a program means or a program package or such like and/or has access to such a program means or package that is embodied for automatic evaluation, where necessary with additional support from operator entries, of the images recorded by the imaging element for the OCT or the IVMRI. In such cases the program means is in a position to determine the scar tissue, which is present for example after a coronary infarction or tissue areas in which conduction path damage is evident. Image processing algorithms are used for this purpose for example which typically allow edge detection or allow deviations in structure in specific areas of an image or patterns to be detected. A comparison with data of a database can also be performed.

Furthermore the control device can be embodied for presentation of at least one recorded image of at least one imaging element and/or for presentation of identified scar tissue and/or of identified tissue with conduction path damage by means of at least one program means. In such cases the information can for example be displayed on a monitor and/or a screen or on a monitor wall assigned to the control device and such like. The display of the identified scar tissue or the area of tissue to be treated and if necessary of an environment makes it possible to check the execution of the ablation directly during the ablation procedure. This can be done by an operator or also automatically by an image comparison or similar.

Accordingly the control device can advantageously be embodied for presentation of at least one recorded image of at least one imaging element and/or of identified scar tissue and/or of identified tissue with damaged conduction paths in real time. This makes it possible to observe the ablation procedure with direct image support and depending on the recorded images of the one or more imaging elements, to guide the ablation, if necessary at least partly automatically.

The myocardial tissue ablation device can be embodied, especially by means of a control device, for a three-dimensional imaging for rotation of at least one imaging element and for simultaneously withdrawal and/or advance of the imaging element and/or of the catheter. For example an OCT sensor or another OCT element can be rotated for creating layer images and simultaneously withdrawn or advanced, with this withdrawal or advance and also the rotation able to be undertaken fully automatically by a control device or undertaken with operator support, in order to create three-dimensional images in this way. This makes an anatomical assignment of the infarction tissue or of the tissue to be treated possible. Accordingly an IVMRI sensor or another imaging element for the IVMRI can be rotated and simultaneously withdrawn or advanced.

At least one position sensor element can be provided in the region of a tip of the catheter. Such a position sensor device makes it possible to identify and locate infarction tissue or tissue with conduction path damage, so that an ablation can be carried out precisely in this area. Position sensors can also be used for example, in connection with IVMRI imaging elements, in order to avoid movement artifacts of a non-exact three-dimensional presentation.

At least one position sensor element can be an electromagnetic or an ultrasound-based position sensor element. Naturally combinations of different sensor elements, for example a number of sensors based on ultrasound combined with a number of sensors based on electromagnetics, can be used in order for example to make possible a redundant failsafe position detection at any time.

At least one position sensor element in the region of the tip of the catheter can be a transmitter, assigned to at least one receiver outside the body of the patient, and/or at least one position sensor element in the region of the tip of the catheter can be a receiver which is assigned to at least one transmitter outside the body of the patient. Accordingly for example the electromagnetic transmitter or alternately the electromagnetic receiver can be arranged in the catheter. The corresponding receiver or in the second case the transmitter are accommodated outside the body. In such cases at least one transmitter is assigned to a receiver or conversely a receiver is assigned to a transmitter, so that position finding within the space is possible. In certain cases however it can also be sufficient, to assign two transmitters to a receiver or vice versa, if for example the angular relationships are known and immutable.

The accuracy of position finding can be increased by an increased number of transmitter or receiver units in the space. However it should be taken into account that the use of a number of transmitters or receivers is associated with a correspondingly increased computing effort, for example for calculating the position on the part of a control device of the myocardial tissue ablation device.

Furthermore at least one transmitter can be embodied for transmitting in all three spatial directions and/or at least one receiver for receiving in all three spatial directions. This enables the position to be easily found within the three-dimensional space.

In addition the myocardial tissue ablation device can feature at least one x-ray imaging device, especially for creating two-dimensional and/or three-dimensional x-ray images.

An additional x-ray imaging device, with which x-ray images can be created to supplement the OCT or IVMRI images, makes it possible to additionally monitor the ablation method on the basis of x-rays.

The myocardial tissue ablation device, especially a control device of the myocardial tissue ablation device can for registration and/or fusing of recorded images of the x-ray imaging device with recorded images of at least one imaging element based on optical coherence tomography and/or intravascular magnetic resonance imaging. For example three-dimensional OCT images or three-dimensional IVMRI-images can also be registered and advantageously be fused with two-dimensional or three-dimensional x-ray images of the x-ray imaging system. The fused images allow guidance of the ablation catheter under additional two-dimensional or three-dimensional x-ray control, for example with images of a system for cardiac computer tomography or of a correspondingly embodied angiography system and similar.

The myocardial tissue ablation device, especially a control device of the myocardial tissue ablation device, can thus be embodied for guidance of the catheter as a function of x-ray images of the x-ray imaging system.

In addition the myocardial tissue ablation device can feature at least one means for magnetic and/or mechanical navigation of the catheter. The use of such a, for example magnetic, navigation system enables a previously usual guide wire or a separate guide catheter to be dispensed with.

As a means for magnetic navigation at least one magnet on the catheter and/or at least one magnetic field creation means outside the catheter can be provided. The catheter can also be provided with magnets which make it possible for the catheter to be controlled and driven by an external magnetic field.

In such cases at least one permanent magnet and/or at least one electromagnet can be provided as the magnet on the catheter and/or at least one coil of a receive antenna equipped with a ferrite core can be provided as position sensor element. The catheter can also feature one or more permanent magnets for example. Furthermore, as an alternative or in addition, electromagnets can be attached to the catheter.

In addition it is possible to provide coils of receive antennas for a position sensor system with ferrite cores, so that with a appropriate dimensioning of these coils such a unit can optionally be used as a receive antenna or as an electromagnet. With activation as an electromagnet magnetic navigation is thus possible. Naturally combinations of these different magnet systems are conceivable for magnetic navigation.

If necessary, as a means for mechanical navigation, at least one pulling device inserted into the catheter can be provided as an alternative or in addition to magnetic navigation means. Such a pulling device is preferably inserted in advance into the catheter and makes it possible via corresponding pull wires or similar pulling facilities to explicitly bend specific sections, preferably the tip or regions at the tip of the catheter, and thus move them into a desired position or location.

Furthermore at least one pressure and/or temperature sensor can be arranged in the region of the tip of the catheter. These and where necessary further sensors, not alluded to specifically here, for recording physical, chemical or physiological data, make it possible to observe specific parameters, preferably parameters relevant for ablation, during the ablation procedure and if necessary, e.g. if limit values that are present in the control device are exceeded, to take measures automatically or with operator support. Such measures can be a brief interruption of the ablation process at critical temperature values and similar.

In addition the invention relates to a catheter for a myocardial tissue ablation device for treatment of cardiac arrhythmias by ablation of myocardial tissue in a patient, i.e. a myocardial tissue ablation device catheter, especially for a myocardial tissue ablation device as described above. This catheter is characterized by being an integrated unit embodied with at least one myocardial tissue ablation means and at least one imaging element based on optical coherence tomography and/or intravascular magnetic resonance imaging. In this case the catheter, as already demonstrated, can feature high-frequency ablation means, especially a high-frequency coil, with which an ablation of tissue which disturbs the heart conduction path is possible.

In addition the invention relates to a method for creation and/or evaluation of at least one recorded image within the framework of a treatment of myocardial disturbances through ablation of myocardial tissue in a patient by means of a myocardial tissue ablation device, especially by means of a myocardial tissue ablation device as previously described. In this method the at least one recorded image is created by means of a catheter embodied as an integrated unit with at least one myocardial tissue ablation means and at least one imaging element based on optical coherence tomography and/or intravascular magnetic resonance imaging and/or at least one created image is automatically evaluated on the basis of optical coherence tomography and/or intravascular magnetic resonance imaging by a control device of the myocardial tissue ablation device and/or registered and/or fused with at least one recorded image of an x-ray imaging system of the myocardial tissue ablation device.

In this case the method does not relate to the ablation procedure itself but merely to obtaining the image data, i.e. physical measurement data, and its subsequent evaluation, for example by a control device, in order preferably automatically, i.e. also without direct involvement of the doctor, to be able to identify scar tissue after an infarction.

The identified tissue then subsequently or in parallel (especially with real time imaging) allows the actual ablation process to be carried out, in which, for example, under the supervision of a doctor, an advantageous automatic or at least partly automatic catheter guidance or operation of the ablation by means of the catheter is carried out.

The inventive method, which relates to the creation and evaluation of the images of a myocardial tissue ablation device with a catheter with corresponding imaging element, can for example be used within the framework of the treatment of ventricular tachycardias and other cardiac arrhythmias.

To this end for example the patient is first positioned on a treatment table, after which an OCT ablation catheter or an IVMRI ablation catheter is introduced in accordance with the invention intravenously. In the event of the catheter featuring an IVMRI imaging element, an MR contrast medium can be injected if necessary the patient can be highlighted in some other way. This can for example involve Gadolinium. The catheter with the imaging elements or an imaging element can for example be introduced via the aorta into the left ventricle. Subsequently OCT or IVMRI images can be created. Furthermore, if a corresponding x-ray device of the myocardial tissue ablation device is present, two-dimensional and/or three-dimensional x-ray images can be recorded in parallel or in advance of or after an ablation. For example processing of the image data in relation to a detection or presentation of soft tissue which can be undertaken with the aid of a synchronization of the recording operation by an electrocardiogram is conceivable.

The OCT, IVMRI and where necessary x-ray images can be fused to enable them in this way to be assigned precisely to scar tissue and the anatomy in the area of the heart chambers.

Subsequently the actual ablation procedure can be conducted, within the framework of which the regions in the ventricle that exhibit the undesired electrophysiological activity can be burned or sclerosed. In such cases the guidance of the ablation catheter is based for example on the fused OCT and x-ray images or fused IVMRI and x-ray images.

Subsequently an OCT image or an IVMRI image and/or a mapping investigation can be undertaken, i.e. an investigation based on the electrophysiological potentials in the heart, in order to check the success of the ablation in this manner.

With a successful ablation the patient can be moved. If the ablation has not yet been successful, the previous steps can be repeated if necessary.

During the ablation a temperature or pressure sensor can be used to monitor the temperature and the pressure in the area of the heart or vessel to be treated. In a similar manner an ablation is possible in the treatment of tumors and metastases.

Thus a simple procedure without multiple introduction or new removal of individual catheters for example is possible for ablation and imaging. The ablation of the tissue parts can be undertaken rapidly and safely. The image support, especially in real time, means that there is also a reduced risk for the patient.

### BRIEF DESCRIPTION OF THE DRAWINGS

Further advantages, features and details of the invention emerge with reference to the following exemplary embodiments and also from the drawings. The figures show:

FIG. 1 an inventive myocardial tissue ablation device with a high-frequency ablation coil and an IVMRI sensor arranged behind the high-frequency ablation coil,

FIG. 2 an inventive myocardial tissue ablation device with a high-frequency ablation coil and an IVMRI sensor arranged in front of the high-frequency ablation coil,

FIG. 3 an inventive catheter with an OCT sensor as well as magnetic sensors,

FIG. 4 an inventive myocardial tissue ablation device with a high-frequency ablation coil with an opening for passing an imaging element through it,

FIG. 5 a diagram of scar tissue for an ablation, and

FIG. 6 a diagram for reading out measurement data for an inventive myocardial tissue ablation device.

### DETAILED DESCRIPTION OF THE INVENTION

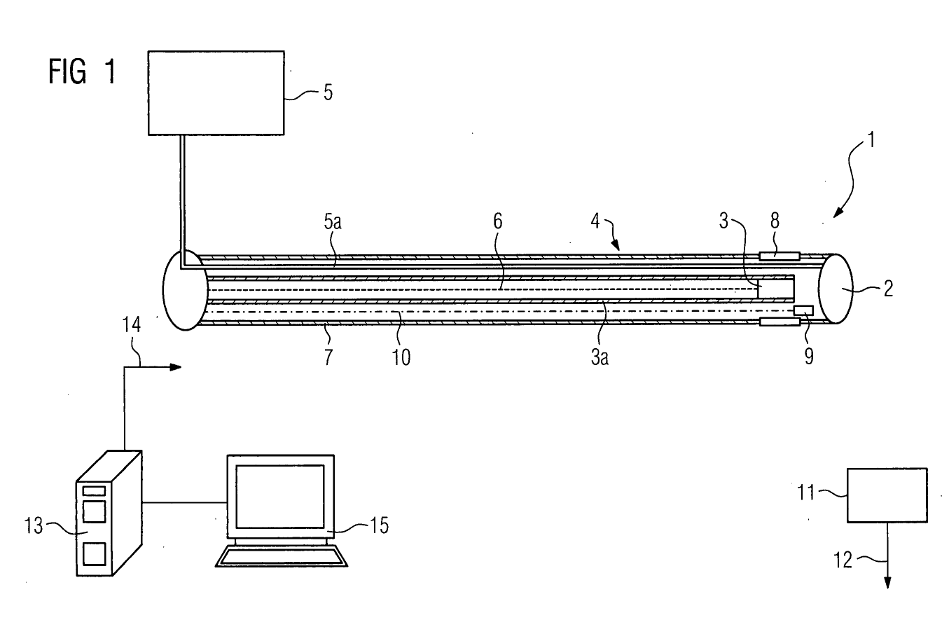

FIG. 1 shows an inventive myocardial tissue ablation device 1 having a high-frequency ablation coil 2 for ablation of tissue with conduction path damage. Arranged behind the high-frequency ablation coil 2 is an IVMRI sensor 3, which, like the high-frequency ablation coil, is integrated into a catheter 4 of the myocardial tissue ablation device. The catheter 4 also features not only myocardial ablation means in the form of the high-frequency-ablation coil 2, which is connected to the high-frequency-source 5 for creating high-frequency IR radiation via a line 5a, but also an imaging element, here in the form of the IVMRI sensor 3, with the associated MRI signal line 6. Furthermore an IVMRI lumen 3a is provided in the catheter 4 for the IVMRI sensor.

In addition the catheter 4 is provided with a catheter shell 7, into which the individual components are fitted.

To make trouble-free magnetic resonance imaging possible, the catheter 4 also features a window 8 transparent for IVMRI.

Magnetic sensors 9 are additionally provided in the region of a tip of the catheter 4, with said sensors being used for magnetic navigation as well as for position detection. In the case shown the transmitters, to which signal lines 10 lead, are provided in the catheter 4 as magnetic sensors 9. In this case a corresponding receiver system 11 is thus arranged outside the body of the patient.

This has an interface to a position detection unit not shown in any greater detail here, as depicted by the arrow 12.

In other exemplary embodiments the arrangement of the transmitter or receiver in the catheter or outside the body of the patient can be embodied entirely the other way round or also mixed.

Furthermore the myocardial tissue ablation device 1 features a control device 13 via which the ablation and the imaging, as indicated here by the arrow 14, can be controlled automatically or if necessary with operator support. The control device 13 is connected to a screen 15, on which images such as those recorded by the IVMRI sensor 3 or images corresponding to the data determined by this sensor can be displayed. Also shown in the images if required is any identified tissue with damaged conduction paths which was determined from the images with the aid of a corresponding program means which is stored on the control device 13, for a real time support of the ablation.

An ablation of myocardial tissue can thus be performed entirely with a myocardial tissue ablation device 1 with a single catheter 4, without for example any image monitoring or removal or re-introduction of an ablation catheter or of a separate imaging catheter being required.

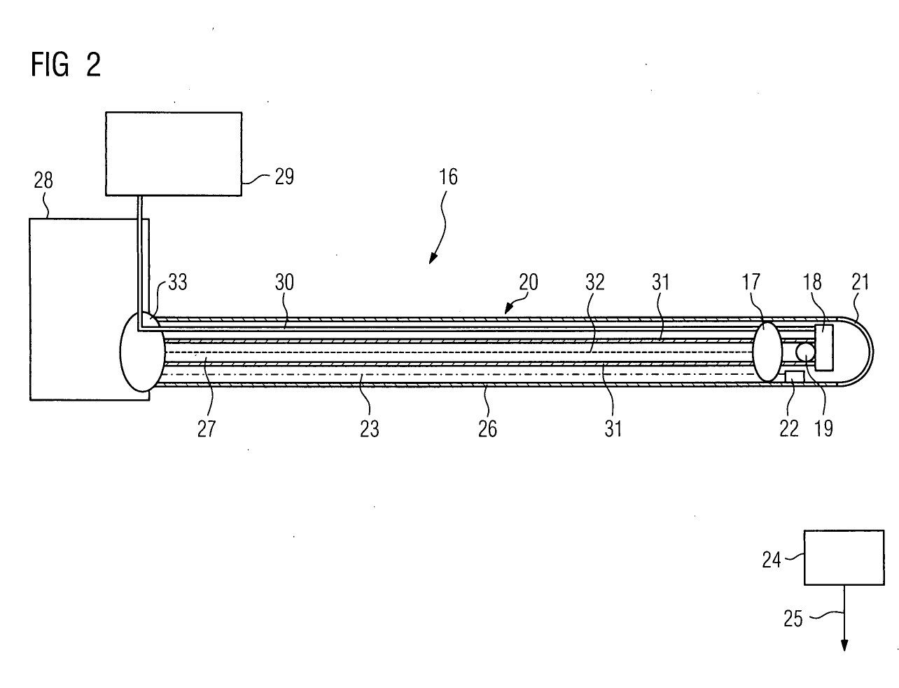

FIG. 2 shows an inventive myocardial ablation device 16 with a high-frequency ablation coil 17 and an IVMRI sensor 18 arranged in front of the high-frequency ablation coil 17. Furthermore a 90° articulated drive 19 is provided to allow optimum imaging.

The catheter 20 with the high-frequency-ablation coil 17 as well as the IVMRI sensor 18 has in its forward area a window 21 transparent for IVMRI, to allows an optimum recording of image data in the region of the tip of the catheter 20 or in a region lying in front of this (also to the side).

In addition magnetic sensors 22 as antennas with associated signal lines 23 and a corresponding position sensor or detector 24 outside the body, i.e. a transmitter or receiver system, are again provided. This position sensor or detector 24 has an interface to a position detection unit which is not shown in any greater detail here, as depicted by the arrow 25. The catheter shell of the catheter 20 is labeled with the reference number 26.

The catheter 20 features a lumen 27 with the boundaries 31 for the IVMRI sensor 18 and the associated MRI signal lines 32 as well as a drive shaft not shown in any greater detail, with the signaling or mechanical Interface and the drive unit as depicted by the box 28 making it possible to drive the catheter 20 or its components.

The high-frequency energy for the high-frequency ablation coil 17 is generated via a high-frequency source 29 which is connected for this purpose via a line 30 to the high-frequency ablation coil 17. A further line not shown is used for activation of the 90-degree articulated drive 19. The connections are made possible via a coupling 33.

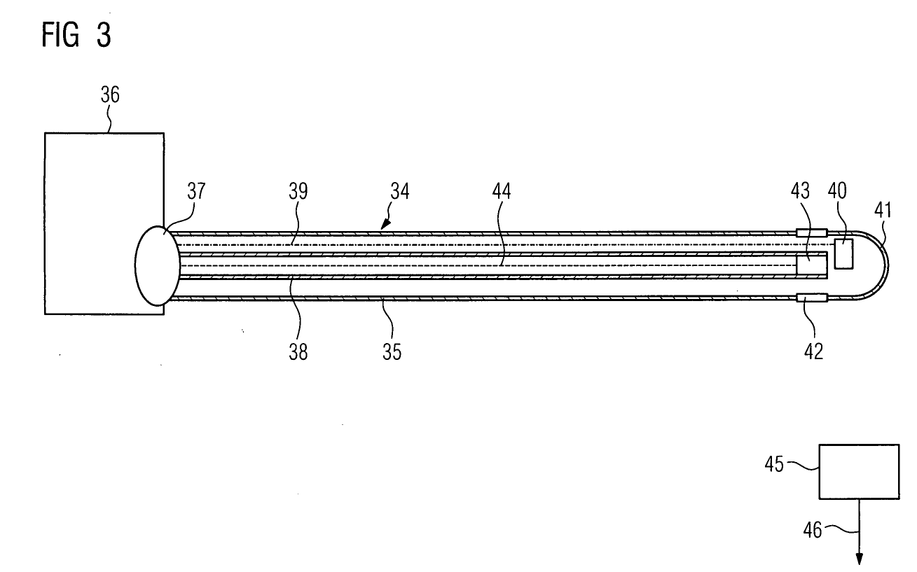

FIG. 3 shows an inventive catheter 34 with a catheter shell 35 as well as an assigned interface for signals or mechanical effects and with an assigned drive unit for the catheter 34 as depicted by box 36. The connection coupling is labeled with the reference number 37. Lines such as signal lines 39 for antennas of a position sensor detection system the magnetic sensors 40 at the tip of the catheter 34 are routed in the catheter sleeve 35 of the catheter 34. The magnetic sensors 40 also serve as coils provided with ferrite cores of receive antennas in the catheter 34 both as receive antennas and also as electromagnets, so that these can be used both for position detection and for an activation as electromagnet simultaneously for magnetic navigation. The rounded tip of the catheter is labeled with the reference number 41.

Furthermore the catheter 34 features a window 42 transparent for OCT close to the rounded tip 41 of the catheter 34. This window 42 transparent for OCT enables the exit of temporally short-coherence light for OCT imaging. For imaging with the aid of optical coherence tomography the catheter 34, as well as the OCT sensor 43, also features an OCT glass fiber 44 which is routed in the OCT lumen 38.

To make position detection possible, transmitters 45 outside the body of the patient or separate from catheter 34 are also provided, which in accordance with the arrow 46 are linked to a position detection unit not shown in any greater detail.

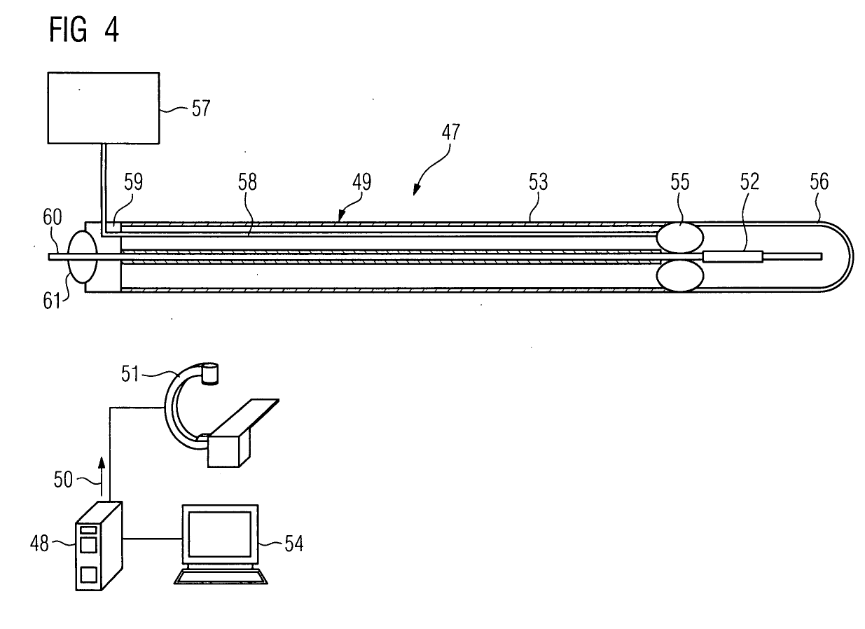

FIG. 4 shows an inventive myocardial ablation device 47 with a control device 48 for activation of a catheter 49 as depicted by the arrow 50. Furthermore the myocardial tissue ablation device 47 features an x-ray imaging device 51, with which, in addition to image recording in accordance with the method of optical coherence tomography with the OCT sensor 52, recorded images can be created at the tip of the catheter 53 of the myocardial tissue ablation device 47. These recorded images are registered and fused jointly with the OCT images of the OCT sensor 52. The recorded images, with any scar tissue or tissue with damaged conductor paths which may have been identified by the control device 48, are shown on an image display device 54, so as to make it possible to monitor an ablation procedure with the aid of the high-frequency ablation coil 55 at the tip of the catheter 53 in real time. The catheter 53 further features a transparent catheter tip 56 which allows light to escape for optical coherence tomography.

The high-frequency energy for the high-frequency-ablation coil 55 is created with the aid of a high-frequency source 57 and transmitted via the line 58 to the high-frequency ablation coil 55. Furthermore the catheter 53 has a mechanical connection system 59 to allow the required connections. The lumen for introduction or passage of the OCT element or of the elements for position detection not shown in any greater detail here is labeled with the reference symbol 60. A rotational coupling 61 in conjunction with the mechanical connection system 59 allows connection of the catheter 53.

The high-frequency ablation coil 55 can, in order to make imaging in front of the high-frequency ablation coil 55 possible, have an opening as shown here, through which the OCT sensor 52 can be pushed and if necessary withdrawn again.

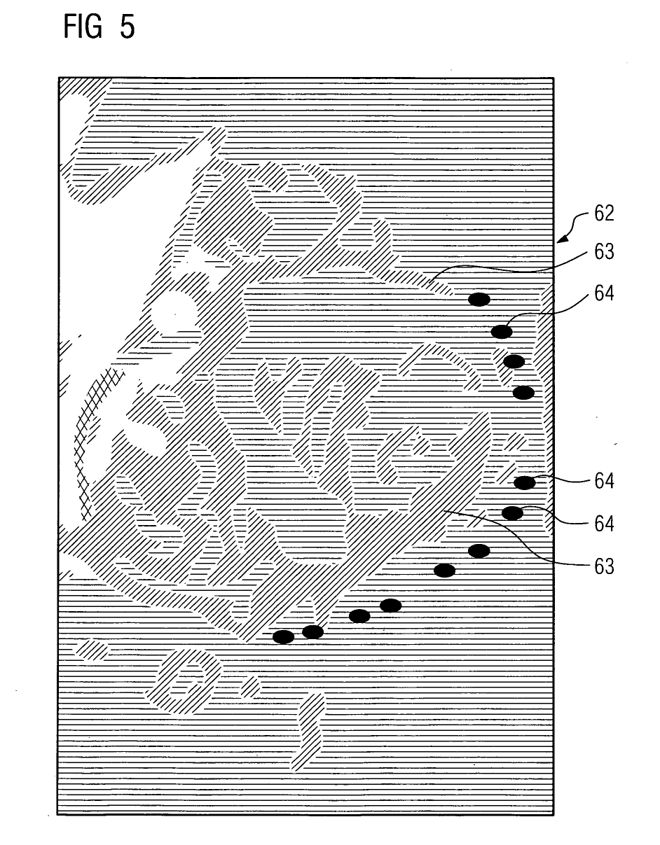

FIG. 5 shows a diagram 62 which includes scar tissue 63 as well as possible ablation points 64 for myocardial tissue ablation. This image 62 is displayed on a screen of an inventive myocardial tissue ablation device which is not shown in any greater detail here, with the image advantageously being displayed in real time, in order to make optimum guidance of the ablation possible. Thus a safer and more rapid ablation of tissue with conduction path damage in the myocardium can be undertaken by the catheter of an inventive myocardial tissue ablation device embodied as an integrated unit with at least one imaging element and at least one myocardial tissue ablation device at a far lower risk to the patient.

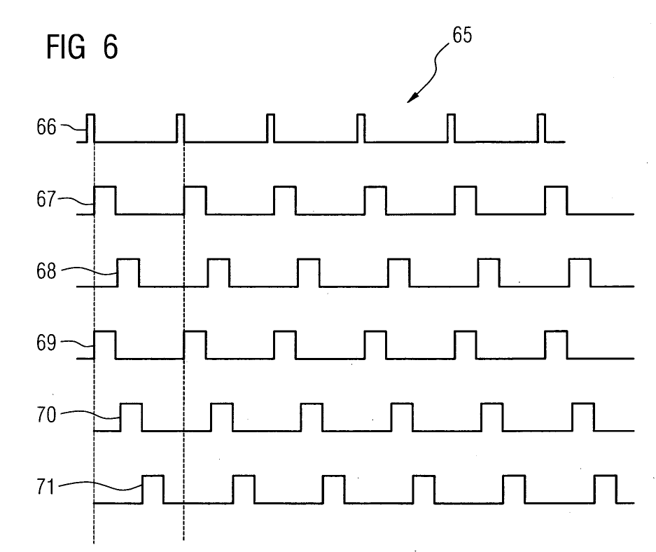

FIG. 6 finally shows a diagram 65 for reading out the measurement data for a myocardial tissue ablation device as claimed in the invention. In this case the different sensors are read out offset in time and clocked for the synchronized read out method. Here the upper curve 66 shows the system clock and the curve 67 arranged below it the switching on of the x-ray radiation of an x-ray imaging device of the inventive myocardial tissue ablation device, which is followed in time by the reading out of the x-ray detector in accordance with curve 68.

The activity times of magnetic positioning are shown by curve 69, with the magnetic positioning being active if the x-ray radiation in accordance with curve 67 is also switched on.

The reading out of IVMRI data in accordance with curve 70 takes place at the same time as the reading out of the x-ray detector in accordance with curve 68. An OCT readout could be undertaken accordingly. Furthermore the myocardial tissue ablation devices have means for ECG recording or measuring the respiration of the patient, the data of which is read out in accordance with curve 71, i.e. after the reading out of the x-ray detector in accordance with curve 68 or the reading out of the data of the IVMRI sensor in accordance with curve 70.

1.-21. (canceled)

22. A myocardial tissue ablation device for a treatment of a cardiac arrhythmia in a patient, comprising:
a myocardial tissue ablation device integrated in a catheter that ablates a myocardial tissue of the patient; and
an imaging element integrated in the catheter that records an image of the patient during the treatment.

23. The myocardial tissue ablation device as claimed in claim 22, wherein the myocardial tissue ablation device emits energy that is selected from the group consisting of: high-frequency, ultrasound, cold, and heat.

24. The myocardial tissue ablation device as claimed in claim 22, wherein the myocardial tissue ablation device comprises a control device.

25. The myocardial tissue ablation device as claimed in claim 24, wherein the control device automatically evaluates and presents the image recorded by the imaging element and identifies and presents a scar tissue or a tissue with a damaged conduction path in real time.

26. The myocardial tissue ablation device as claimed in claim 24, wherein the control device rotates the imaging element and simultaneous withdrawals or advances the imaging element or the catheter.

27. The myocardial tissue ablation device as claimed in claim 24, further comprising an x-ray imaging device for creating an x-ray image of the patient.

28. The myocardial tissue ablation device as claimed in claim 27, wherein the control device registers or fuses the x-ray image with the image recorded by the imaging element.

29. The myocardial tissue ablation device as claimed in claim 27, wherein the control device guides the catheter based on the x-ray image.

30. The myocardial tissue ablation device as claimed in claim 22, further comprising a position sensor element in a region of a tip of the catheter.

31. The myocardial tissue ablation device as claimed in claim 30, wherein the position sensor element is an electromagnetic or an ultrasound-based position sensor element.

32. The myocardial tissue ablation device as claimed in claim 30, wherein the position sensor element is a coil of a receive antenna equipped with a ferrite core.

33. The myocardial tissue ablation device as claimed in claim 30, wherein the position sensor element is a transmitter that is assigned to a receiver outside a body of the patient for transmitting data in three spatial directions.

34. The myocardial tissue ablation device as claimed in claim 30, wherein the position sensor element is a receiver that is assigned to a transmitter outside a body of the patient for receiving data in three spatial directions.

35. The myocardial tissue ablation device as claimed in claim 22, wherein the catheter comprises a magnet or a magnetic field creation device provided outside the catheter for a magnetic navigation of the catheter.

36. The myocardial tissue ablation device as claimed in claim 35, wherein the magnet is a permanent magnet or an electromagnet.

37. The myocardial tissue ablation device as claimed in claim 22, wherein a pulling device is inserted into the catheter for a mechanical navigation of the catheter.

38. The myocardial tissue ablation device as claimed in claim 22, wherein a pressure or a temperature sensor is arranged in a region of a tip of the catheter.

39. The myocardial tissue ablation device as claimed in claim 22, wherein the imaging element is an optical coherence tomographic imaging element or an intravascular magnetic resonance imaging element.

40. A catheter for a treatment of a cardiac arrhythmia in a patient, comprising:
a myocardial tissue ablation device integrated in the catheter that ablates a myocardial tissue of the patient; and
an imaging element integrated in the catheter that records an image of the patient during the treatment.

41. A method for treating a cardiac arrhythmia in a patient, comprising:
recording an image of the patient by an imaging element integrated in a catheter of a myocardial tissue ablation device;
automatically evaluating the image by a control device of the myocardial tissue ablation; and
ablating a myocardial tissue based on the evaluated image.
